(19) United States
(12) Patent Application Publication
(10) Pub. No.: US 2005/0085752 A1
(43) Pub. Date: Apr. 21, 2005

(54) Scrotal support surgical positioner

(76) Inventor: Haskell L. Sells II, Raleigh, NC (US)

Correspondence Address: Haskell Lee Sells II, 3900 Yew Circle, Raleigh, NC, 27612, US

(21) Appl. No.: 10/687,033

(22) Filed: Oct. 17, 2003

(51) Int. Cl. A61F 5/00

(52) U.S. Cl. 602/6

(57) ABSTRACT

In the healthcare field there are specific swellings of the male testicles generally referred to as “Scrotal swelling” or “Testicular enlargement”. These swellings are generally caused by: injury, such as a pelvic injury from a fall or motorcycle accident; certain medical treatments; surgery in the genital area; medical conditions such as Orchitis (infection), hernias, congestive heart failure, Hydrocele (fluid accumulation in sack along the spermatic cord within the scrotum) and Testicular torsion; or, a combination of two or more of these causes. Scrotal swelling can cause discomfort to the patient due to the weight of the swelling. In some cases the swelling is so severe that Scrotal skin and/or surrounding skin may tear or breakdown. The treatment of these swellings can be as simple as ice packs and rest, to surgery and medication. Since this enlargement can be extremely uncomfortable due to it's weight and pull on the skin (potentially tearing the skin or causing skin breakdown), it may be suggested that the patient wear a “jock strap support” when up walking. When sitting or lying down it is suggested that rolled up towels be placed underneath the Scrotum to relieve the pull on the skin.

### CROSS REFERENCE TO REEALTED APPLICATIONS

Related application Ser. No. 08/411,084
U.S. Pat. No. 6,488,031 B1
Date Issued: Dec. 3,2002

### FIELD OF THE INVENTION

In the past, scrotal support for a swollen Scrotum (also called enlarged Testicles) has primarily consisted of a “jock strap support” for an ambulatory patient and rolled up towels, for the patient who is sitting or lying down. The problem with these “manufactured products” include: inappropriate use for a swollen Scrotum, i.e., most “jock strap supports” are not intended for a swollen Scrotum, but rather protection during athletic endeavors and are not appropriate for the patient who is sitting or lying down; rolled up towels do not have consistency of size, shape, density or design and are not intended to be scrotal supports. Some of their problems would be lack of an adequate support area, inability to keep in place, inability to maintain a desired shape, wrong size for needed purpose.

An appropriate positioner for the patient when sitting or lying down should be supportive, comfortable, and well accepted by the patient. It should provide the healthcare personnel a positioner which is easy to place under a swollen Scrotum and support the Scrotum comfortably. It should help to prevent the skin from tearing or breaking down due to the weight and pull of the enlarged Scrotum. It should provide immediate visual assessment of the patient's Scrotum and skin condition. It should be adjustable so that healthcare personnel could use it appropriately for patients of different body types and different “swelling related” size problems. It should be a “one positioner fits all”. It should be of a size and shape for easy and comfortable placement under the Scrotum without discomfort to the patient; it should be of a consistent material for continuity of care over time.

### OBJECT

It is, therefore, an object of the present invention to provide a new and novel scrotal support positioner for a patient when sitting or lying down. This new positioner will help relieve discomfort caused by the weight, and the accompanying pulling of a swollen Scrotum, by helping to support the Scrotum and to help prevent skin tearing and skin breakdown.

Another object of the present invention is to make this positioner easily identifiable by the healthcare staff as to its purpose; to provide immediate visual assessment of the Scrotum and surrounding skin; to be adjustable so that “one positioner fits all”; to be easily placed and comfortable for the patient; to be well accepted by the patient; to be of a consistent material for continuity of care.

### SUMMARY OF THE INVENTION

A Scrotal positioner designed to assist in the appropriate positioning of a sitting or lying (down) patient whose enlarged Scrotum has been recognized as having the potential for skin tearing, skin breakdown and discomfort (from weight, size and the accompanying pulling) is placed under the Scrotum. The positioner positions the patient's Scrotum in such a manner as to help relieve discomfort and help prevent skin tearing and skin breakdown. The positioner is passive in that it is strategically placed by a nurse (or other healthcare professional) so as to best benefit the patient. It will provide immediate visual assessment of the Scrotum and surrounding skin; it will be easily recognizable by the healthcare staff as intended for the Scrotum. The positioner may be used as is or adjusted for height and length so that “one size fits all”.

### DETAILED DESCRIPTION OF PRODUCT AND DRAWINGS

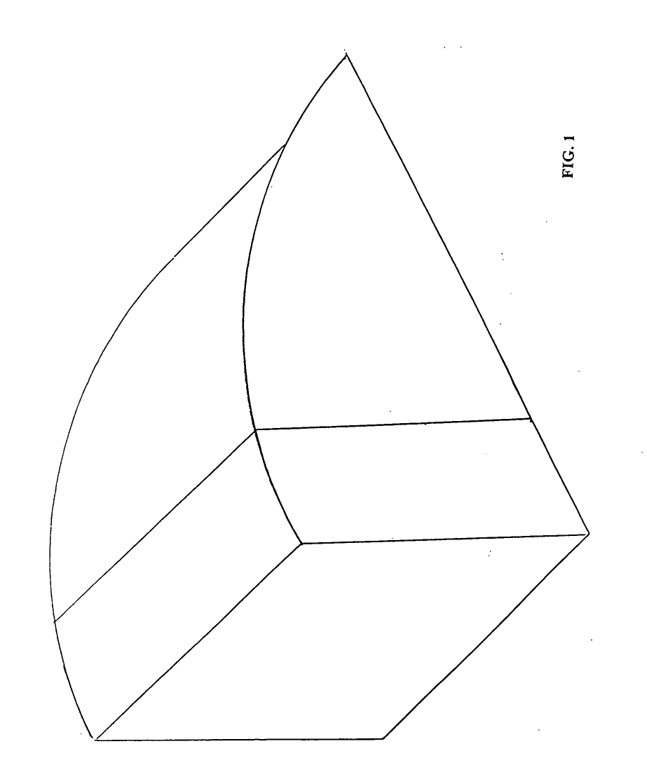

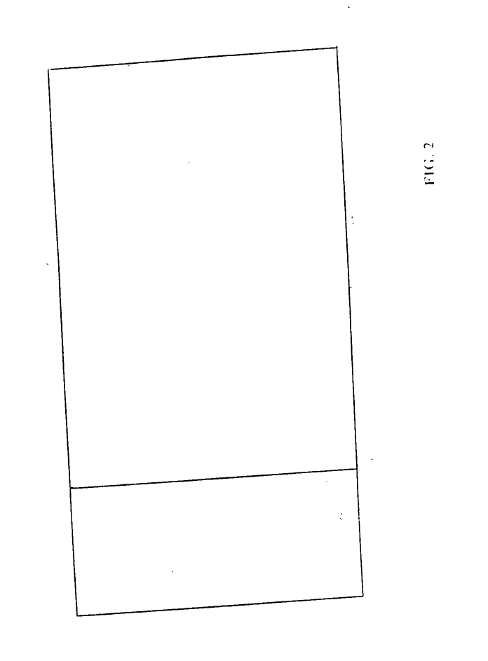

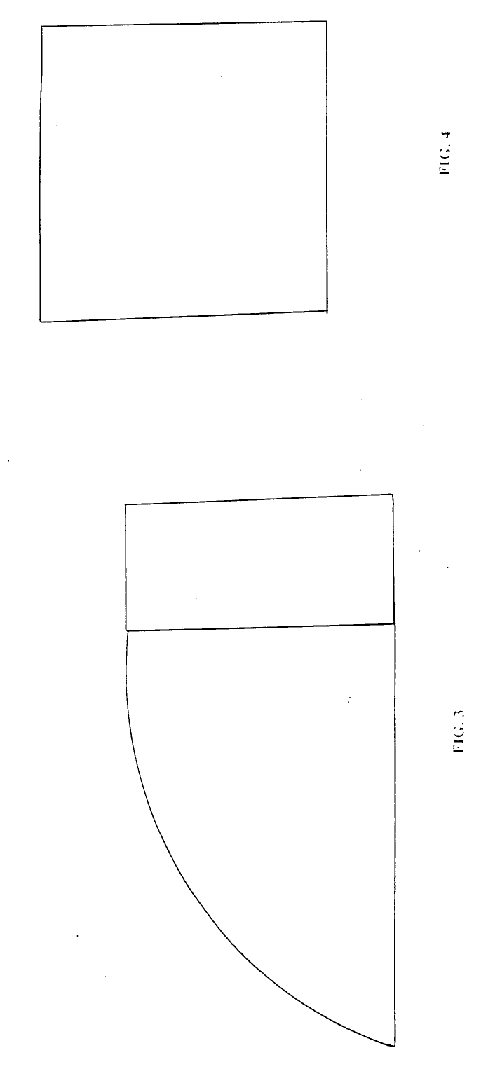

The invention is the design of the positions 10, in that the positioner is the first uniform Scrotal positioner; the first to be designed for use solely with the Scrotum. While the design could be made from a variety of materials (such as various densities of foam, cotton wadding, cellulose, or any other material that can be shaped, stuffed, compressed or otherwise formed to create this shape), the present design is composed of a foam block which is of a 2.2 density and an indention load deflection of 36 (See FIGS. 1 and 3). The foam block is 6.5 inches long (comprised of two smaller foam blocks, one said block being 5 inches long, the other said block being 1.5 inches long, joined in such a manner as to allow the 1.5 inch block to fold under the 5 inch block), 3.5 inches wide, 3.5 inches tall with a curved angle starting at 58 degrees from the front of the positioner (the angle flattens as it nears the back) presenting an arc shape as it reaches the back. (See FIG. 2). While the current invention is of this size and cut, any other sizes, whether larger or smaller could be used. Different combinations could be used from a single one block cut, to different two block cuts, to three block cuts or multiple block cuts. Different heights, widths, lengths, curved angles or no curved angles at all could be used to make this positioner.

The material used to cover this positioner is a rip-stop nylon, but any material (such as plastic, rubber, cotton, polyester or others) could be used, or the positioner could be uncovered.

### DETAILED DESCRIPTION OF PRODUCT AND DRAWING

Brief Description of the Drawings

FIG. 1 is an isometric view of the positioner.

FIG. 2 is a top plan view of the positioner.

FIG. 3 is a side elevation of FIG. 1

FIG. 4 is a rear elevation.

1. A therapeutic positioning device for preventing or assisting in the healing of skin tears and skin breakdown of a swollen Scrotum for a patient who is sitting or lying down, comprised of a resilient body, said body being constructed of a foam block, said block is 6.5 inches long (comprised of two smaller foam blocks, one said block being 5 inches long, the other said block being 1.5 inches long, both being joined in such a manner as to allow the 1.5 inch block to fold under the 5 inch block), 3.5 inches wide, 3.5 inches tall with a curved angle of 58 degrees starting from the front of the positioner (the angle flattens as it nears the back) presenting an arc shape as it reaches the back.

2. A therapeutic positioning device for support of a swollen Scrotum for a patient who is sitting or lying down, comprised of a resilient body, said body being constructed of a foam block, said block is 6.5 inches long (comprised of two smaller foam blocks, one said block being 5 inches long, the other said block being 1.5 inches long, joined in such a manner as to allow the 1.5 inch block to fold under the 5 inch block), 3.5 inches wide, 3.5 inches tall with a curved angle of 58 degrees starting from the front of the positioner (the angle flattens as it nears the back) presenting an arc shape as it reaches the back.

3. A therapeutic positioning device for the comfort of a swollen Scrotum for a patient who is sitting or lying down, comprised of a resilient body, said body being constructed of a foam block, said block is 6.5 inches long (comprised of two smaller foam blocks, one said block being 5 inches long, the other said block being 1.5 inches long, joined in such a manner that the 1.5 inch block can fold under the 5 inch block), 3.5 inches wide, 3.5 inches tall with a curved angle of 58 degrees starting from the front of the positioner (the angle flattens as it nears the back) presenting an arc shape as it reaches the back.

4. An adjustable positioning device for the support and comfort of varying sizes of swollen Scrotum patients when those patients are sitting or lying down, comprised of a resilient body, said body constructed of a foam block, said block is 6.5 inches long (comprised of two smaller foam blocks, one said block being 5 inches long, the other said block being 1.5 inches, joined in such a manner that the 1.5 inch block can fold under the 5 inch block), 3.5 inches wide, 3.5 inches tall with a curved angle of 58 degrees starting from the front of the positioner (the angle flattens as it nears the back) presenting an arc shape as it reaches the back.
